(19) United States
(12) Patent Application Publication
(10) Pub. No.: US 2022/0133270 A1
(43) Pub. Date: May 5, 2022

(54) ACCESSORY TECHNOLOGIES FOR ULTRASOUND SYSTEMS

(71) Applicant: Luiz Maracaja, Salem, NC (US)

(72) Inventor: Luiz Maracaja, Salem, NC (US)

(21) Appl. No.: 17/518,619

(22) Filed: Nov. 4, 2021

Related U.S. Application Data
(60) Provisional application No. 63/109,523, filed on Nov. 4, 2020.

(51) Int. Cl. A61B 8/00; A61B 50/33

(52) U.S. Cl. CPC A61B 8/4427; A61B 8/4422; A61B 8/4411; A61B 50/33

(57) ABSTRACT

Certain embodiments are directed to accessories for the use of portable probes, tablets and smartphones during ultrasound guided procedures requiring real-time visualization and sterile precautions. The embodiments include sterile procedural kits allow the placement of tablets or smartphone into sealed compartment that maintain sterility, allow for touch screen adjustments and continuous visualization.

### BACKGROUND

### Field of Invention

The present invention relates to medical field procedures requiring sterile field such as surgery or needle guided interventions that require imaging equipment for guidance.

### Background

Millions of surgeries and venous catheterization procedures are performed daily worldwide and maximum sterility is necessary to prevent infections. Surgical site infections and central line associated infections are related to high morbidity and mortality. These infections can be prevented by proper use sterile practices including disinfecting the skin with antiseptic solution, using sterile surgical instruments, hand hygiene and use of barrier apparatus such as sterile gown, gloves, drapes and covers for non-sterile equipment such as imaging systems. Catheter-related bloodstream infections are common, costly, and potentially lethal. Each year in the United States, central venous catheters may cause an estimated 80,000 catheter-related bloodstream infections and, as a result, up to 28,000 deaths among patients in intensive care units (ICUs).

Central venous catheterization is a medical procedure with potentially serious complications. Important organs and anatomic structures are closely located around the central veins. The wrong placement of the needle can cause several minor and major related complications (e.g., unsuccessful access, pain, discomfort, arterial puncture, arterial dissection, stroke, pneumothorax, hemothorax, air embolus, arrhythmias, catheter knotting, hemothorax, tamponade, chylothorax, brachia, plexus injury, bleeding, air embolism, etc.). The number of unsuccessful attempts is directly related to the risk of complications. The use of ultrasound guidance reduces the risk of complications associated with vascular access. The use of ultrasound for real time imaging guidance remarkably reduces these complications and has become the standard of care for central line catheterization.

The use of ultrasound in the sterile field is currently performed by using a sterile long cover or plastic bag as a barrier for the ultrasound probe and cable that connects the probe with the computer unit of the machine. Since ultrasound guidance for central line a currently considered standard of care, the placement of the cover became an additional necessary step. The materials and supplies needed to perform central line placement procedure are often packaged separately.

The procedure involves several steps and check lists have been created in order to provide the appropriate materials for this placement of a central line. These steps are time consuming and extra personal is frequently needed to handle the supplies for the line placement. Difficulties in having all the materials and enough personnel are exacerbated because central line placement is frequently needed in emergency situations. All the steps and time needed to provide appropriate sterile field and sterile ultrasound probe increase the time needed to perform the procedure. Time limits frequently lead to several problems such as, noncompliance with use of ultrasound, contamination of sterile material, lack of time for planning the best approach and consequentially a less precise placement resulting in potential complications.

Real time ultrasound guidance for interventional procedures require adjustments of direction and depth of the needle insertion in relation to the position of the ultrasound probe. The parameters for the adjustments are based on visualization of ultrasound image in a display screen. The display screen and insertion site are usually located in different physical spaces. The image of the needle insertion in real time give the biofeedback to the proceduralist to make directional changes as the needle is advanced, therefore it is required few cycles of change eyesight from the needle insertion site to the display screen, adjust position and continue the needle insertion.

Physical space is a consideration and potential issue when performing ultrasound guided procedures since a table to open the supply tray(s), ultrasound system, bed for the patient, and room to person performing the procedure and person to assist with the procedure. The large ultrasound systems are placed in carts with wheels and the display screen are located above the cart.

The current smartphones and tablets technology have tremendous computing power and they could be used for several functions including real time imaging during surgical and interventional procedures. The portable ultrasound systems use smartphones or tablets for computing data and display the images. The placement of the display screen close to the insertion site would be ideal for the procedure, however the lack of appropriate sterile barrier and for the tablet or smartphone, probe and cable could put sterility in risk and contaminate the sterile field and cause infections.

There remains a need for more efficient accessories, sterile materials, sterile supplies and procedural kits for the use of smartphones, tablets and imaging devices during needle guided interventions and surgeries.

### SUMMARY

The present invention seeks to provide sterile apparatus for the use of smartphones, tablets and imaging devices during needle guided interventions and surgeries. Embodiments of the invention include materials and devices to be used in conjunction with interventional medical procedures, such as ultrasound guided venous catheterization. Certain embodiments are directed to accessory materials and kits for providing a sterile surface, sterile components (including instruments and sterile dress), and an environment for medical procedures using ultrasound guidance.

In certain aspects a sterile kit or package described herein includes the materials needed to perform a central line placement. The kit is ergonomically designed to minimize risk of infection, minimize personnel required, and minimize time for insertion of central line catheters. The kit comprises a tray with all necessary supplies to for sterile preparation as well as the supplies to perform the procedure.

The procedural kit provides all the necessary sterile supplies for a procedure and organizes those supplies in an as needed order. The design of the kit reduces the need of physical space for both storage and use, as well as localizing all components in one location. The kit reduces amount of manipulation need for opening packages and accessing supplies, which reduces the potential of infection, as well reducing the number of personnel need to prepare for a procedure. The kit provides for sterile preparation of the provider, the ultrasound equipment, and the patient all in one kit.

Certain embodiments described herein are directed to two configurations of sterile packaging kits and a sterile drape specifically designed and configured for the use of tablet or smartphone and portable imaging systems (such as ultrasound probe as an example).

The advantage of giving access to a smart device and imaging capability directly of the procedure drape allows use of several technologies in locations and places such as, prehospital EMS, war zones, remote locations, developing nations. It also would also reduce time and physical space necessary to gather all the equipment and perform the procedure.

The tablet or smartphone with computing unit and screen viewer can be used for functions such as: 1—different modalities of real time imaging. 2—radiology exams such as Xrays, CT scans, Mills, coronary angiography, etc. 3. real time imaging transmission of slides and communication with the pathology lab; 4. transmission of physiologic monitoring 4—real time communication with other specialty providers, or units such as intensive care unit or blood bank, etc.

In addition to the ultrasound imaging the computing unit can be used in real time associated with different sensors and transducers such as infra-red thermal cameras, radiotracers, flow probes, dopplers, x-Rays, endoscopes, radiofrequency probes, EEG, EMG, etc; the tablet/smart phone controller will have capability to control different pieces of equipment such as electrocautery, diathermia, radiofrequency probes, cryoprobes and associated.

Another aspect achieved with current invention and the smart devices is the possibility of wireless transmission of data from the sterile field using different technologies such as WIFI, Bluetooth, LTE, etc.

The drape contains a built in sleeve for the probe and a compartment for the smartphone or tablet where the top face of the upper compartment is made of a transparent and thin material such as polycarbonate sheet that allows visualization and touchscreen manipulation of the device using sterile gloves. The sterile drape contains specific compartments for the tablet or smartphones and ultrasound probe.

The respective compartments allow sterile use of the devices during the procedures. The kit is configured to provide access to the lumen or interior compartments inside the drape for placement of ultrasound probe and the tablet without contaminating the surfaces that require sterility. The access of the interior compartments is done by crossing two openings of same size and shape, one on the top of the package and the second opening on the bottom surface of the drape. The devices are placed inside the drape before the package is fully opened.

The two openings are designed for one-way direction of insertion; therefore, the probe or tablet won't accidently come out or fall out of the internal compartment after insertion. The opening on the bottom of the drape is subsequentially sealed with a sterile plastic film to prevent that any germ in the area of the device placement to reach and contaminate the sterile field.

The tablet and probe, with stay inside the folded drape. After sterile preparation of the surgical field the drape is carried to the surface of the skin and the drape is unfolded according to instructions and labels on the surface of the drape. The unfolded drape will allow the tablet and probe to be in specific position next to the operative field, that can easily be accessed by the person performing the procedure.

The package opening has two separate sealed covers or slots covered by peel away fashion package opening. The first slot opening is directly aligned with the compartment on the bottom of the drape where the probe and the table or smartphone can be inserted.

The second slot or large cover contains necessary items to sterile prepare the person performing the procedure and the patient skin prior to intervention. In certain aspects the second compartment or preparation compartment contains preparative items such as hat, mask, gown, gloves and skin preparation solution(s), e.g., ChloroPrep®.

After appropriate sterile preparation of the proceduralist and the patient, on the bottom of the drape, a sterile cover with adhesive material is located adjacent to the opening for the probe and tablet. Such cover allows sterile seal and cover slot on the drape where the nonsterile phone and probe were inserted. This feature will guaranty that any non-sterile part of the drape that contacted the drape during the placement with be covered by a sterile layer of plastic material.

In certain aspects the package cover is configured so that the ultrasound transducer and tablet can be positioned inside compartment before opening the second sterile compartment. The sterile kit is designed to be used in a certain sequence and can reduce the time needed to perform line placement while allowing the entire procedure to be performed in a sterile fashion by one health care provider.

Once the patient and provider are prepared and the transducer positioned in the transducer cover, the kit shell or container is opened to access the surgical drape and instrument package. The surgical drape is package in such a way so that the transducer can be positioned with respect to the patient and the surgical drape deployed to form a covered field. Once the surgical drape is deployed the instrument package is configured to have one or more sealed compartments that can be accessed by removing or peeling a cover to a compartment. In certain aspects the linear order of the instruments correlates with the steps involved in the intervention procedure.

Thus, to initiate an intervention procedure one would need to obtain the kit, open the slot of the package and insert probe and tablet or smartphone into respective compartments on the drape. The preparation compartment is opened and the person dresses and prepares the patient. Once these preparations are complete the adhesive sterile seal covers the entry of the probe and tablet on the drape. Next the fenestrated drape is deployed and unfolded on the region of interest. The final position for the tablet or smartphone and probe will be adjacent to the fenestration where the procedure will be performed.

Furthermore, the design of the kit provides for efficient ergonomics as components of the kit opens around the equipment used for guiding the procedure and provides the instruments needed in a readily accessible location when those instruments are needed. Embodiments described herein allow fast, precise adjustments in positioning imaging devices and for proving guidance or proper placement of central venous catheter (CVC), also known as central line, in an environment that reduces the risk of infection. A central line is a catheter placed into a large vein in the neck, chest, or groin. A central line is used for administering medication or fluids, obtaining blood samples, hemodynamic monitoring, temporary cardiac pacing, and/or performing hemodialysis. Devices and materials described herein can be used by interventional radiologists, surgeons, anesthesiologists, emergency personnel, and critical care physicians. Embodiments of the kits and devices described herein provide the supplies needed to perform the procedure in a single package. The design of the kit provides for a package opening process that allows for performance of all necessary steps in sterile fashion. The kits and device minimize personal, time and physical space needed to perform the procedure in sterile fashion. The kit can also be used for other intervention and surgery, with variation of the necessary supplies or devices. The kit design and peripherals save time and increase physician compliance with the use of ultrasound protocol. Pre-sterilized medical procedure kits are known and used for various medical procedures. Such sterilized procedure kits are provided with a plurality of components used in connection with an intervention or surgical procedure. Certain embodiments are directed to sterilized surgical kits organized and accessed in a way that maintains a sterile environment or reduces the risk for infection during a procedure, as well reducing the time for initiating and/or performing the procedure. Kits of the invention can be used in conjunction with ultrasound devices and for performing ultrasound guided procedures.

Another embodiment is a mounting system that allows connection between a smartphone and the probe with thread on the surface of mounting to place the redundant connection cable and minimize the risk of contamination during the procedure. Other embodiments also include a respective sterile cover for the mounting system, probe and smartphone.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates the sterile package or tray containing all the sterile supplies needed for a procedure.

FIG. 2 illustrates the peel off the smaller cover, the edges of the small cover contain adhesive material and removal is performed by continuous lift of tab.

FIG. 3 illustrates the sterile insertion of the ultrasound probe (6) and cable (7) into the respective slot (5).

FIG. 4 illustrates the smartphone or tablet with display screen (8) insertion on the slot (4).

FIG. 5 illustrates the probe (6) and the tablet (8) located in separate compartments inside the drape before the opening of the large cover (3).

FIG. 6 illustrates the opening of the large cover (3) of the package or tray (1) giving access to the sterile supplies to perform the procedure and the drape (14).

FIG. 7 illustrates the step after removal of the sterile supplies, the peel away cover (15) is removed and a plastic film (16) with adhesive substance is flipped over to provide sterile cover for the slots (4 & 5) where the probe, cable and tablet viewer were inserted.

FIG. 8 illustrates the adhesive on the plastic film (16) facing the drape and provides complete sterile seal of the slots (4 & 5) and adjacent face where the probe and tablet had contact during the insertion.

FIG. 9 illustrates the drape 14 with surface entirely sterile, being removed from the package or tray (1) by the sterile gloved hands (17) of the health care provider.

FIG. 10 illustrates the drape (14) surface that will be in contract with the patient.

FIG. 11 illustrates the placement and opening of the drape (14) on the patient (22), the fenestration (20) should aim the final expected position of the procedure site (21).

FIG. 12 illustrates the process of unfolding the drape (14).

FIG. 13 illustrates the final position of the drape (14) on the patient (22).

FIG. 14 illustrates the opening the compartment (24) on the drape that will give access to the probe (6), tablet (8), and central line supplies (27).

FIG. 15 illustrates the probe (6) placement on the patient's neck displaying the images of neck vessels on the tablet screen (8).

FIG. 16 illustrates the process of imaging using portable ultrasound probe (6), respective cable (7), and the smartphone used as a display screen or viewer for real time imaging.

FIG. 17 illustrates the parts of mounting system that connects the probe (6), with the ultrasound system.

FIG. 18 illustrates the assembly of probe (6), smartphone (8), and mounting system (30 and 36).

FIG. 19 illustrates the sterile package or tray (40) containing all the sterile supplies needed for a procedure (1).

FIG. 20 illustrates the small cover (42) opening by peeling off from the large cover (41) of the tray (40).

FIG. 21 illustrates the insertion of the assembly probe (6), smartphone and mount (30) inside the package through the package opening.

FIG. 22 illustrates the aligning plate on the back of the mounting system (44), is aligned and attached to the two alignment pins (43).

FIG. 23 illustrates the large cover (41) being peeled off from the package (40) showing the internal components of the package.

FIG. 24 illustrates the open tray (40) with the probe now inside the cover (47) and the top closure (45) in open position.

FIG. 25 illustrates the top cover (45) folded in a half closing the compartment.

FIG. 26 illustrates the clear film (46) that allows visualization and touchscreen manipulation of the smartphone (8) by the provider using sterile gloves (48).

FIG. 27 illustrates an example of real time ultrasound guided procedure where the provider is using sterile gloves (48) holding the assembly with the left hand and the syringe (52) with needle attached (51) with the right hand.

FIG. 28 front view of one example of an ultrasound adaptor device.

FIG. 29 back view of one example of an ultrasound adaptor device.

FIG. 30 top view of one example of an ultrasound adaptor device.

FIG. 31 bottom view of one example of an ultrasound adaptor device.

FIG. 32 side view of one example of an ultrasound adaptor device.

FIG. 33 perspective view of one example of an ultrasound adaptor device.

FIG. 34 side view of one example of an ultrasound adaptor device with viewing device attachment, e.g., smart phone, assembled with an ultrasound probe.

FIG. 35 front view of an ultrasound probe, adaptor device, and a smart phone assembly.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

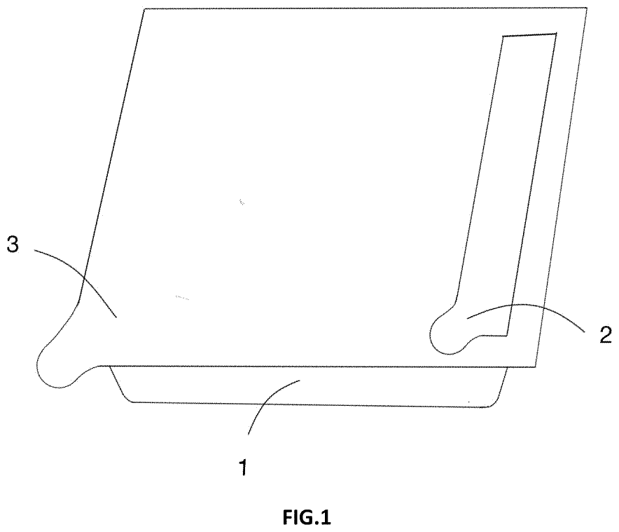

FIG. 1 illustrates the sterile package or tray containing all the sterile supplies needed for the procedure (1), showing two covers, one small (2) covering a smaller area and one large (3) covering the remaining top package or tray.

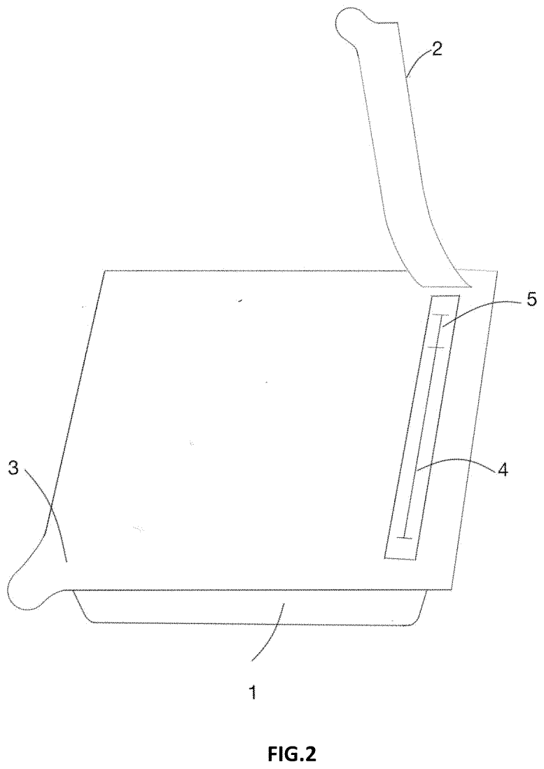

FIG. 2 illustrates the peel off the smaller cover, the edges of the small cover contain adhesive material and removal is performed by continuous lift of tab. The removal will detach the small cove (2), from the large cover (3) and tray (1). Upon removal of the small cover (2) access of two slots for respective insertion of probe (5) and smartphone or tablet viewer (4) become available. The slots will communicate with respective compartment inside the drape.

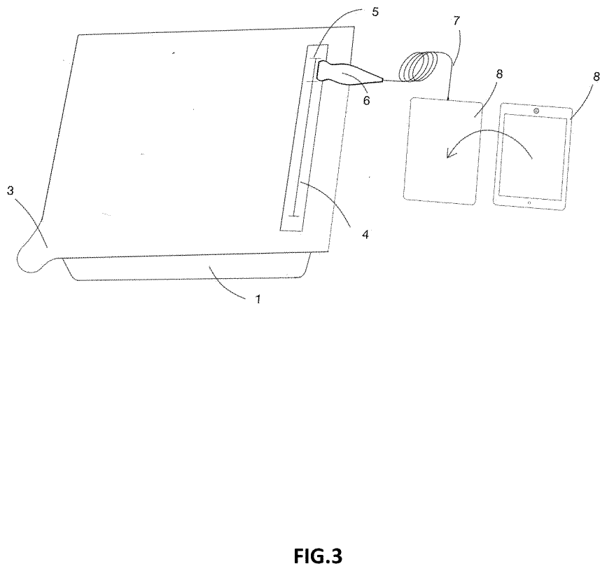

FIG. 3 illustrates the sterile insertion of the ultrasound probe (6) and cable (7) into the respective slot (5). The tablet or viewer display (8) should face down before the insertion on slot respective slot (4).

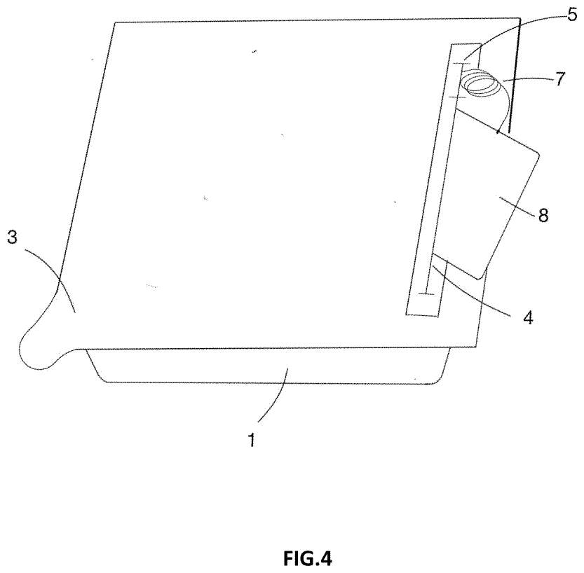

FIG. 4 illustrates the smartphone or tablet with display screen (8) insertion on the slot (4). The insertion of the probe (6) and tablet (8) into the respective slots is performed with the large cover (3) for the tray (1) in place.

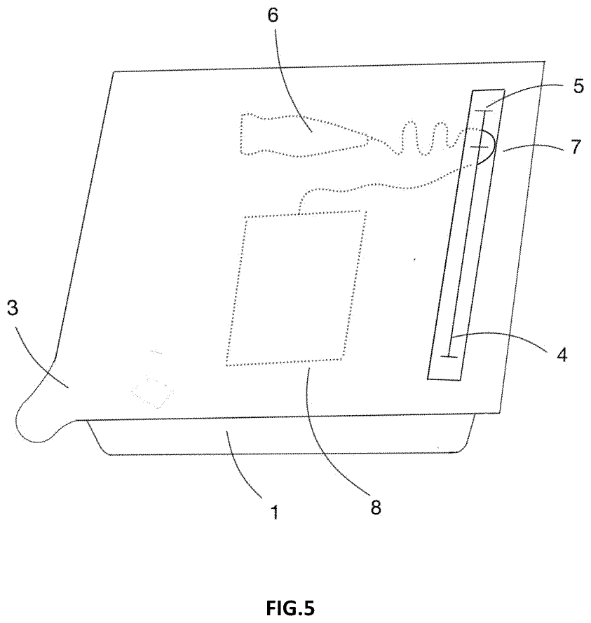

FIG. 5 illustrates the probe (6) and the tablet (8) located in separate compartments inside the drape before the opening of the large cover (3). Prior to using the kit and initiating the procedure any auxiliary equipment's or devices will need to be assembled and/or prepared. In certain aspects an ultrasound probe and tablet or smartphones devices be acquired and prepped for use. In certain aspect ultrasound conducting gel material is positioned inside and outside of the transducer cover in order to enhance ultrasound transmission to the transducer.

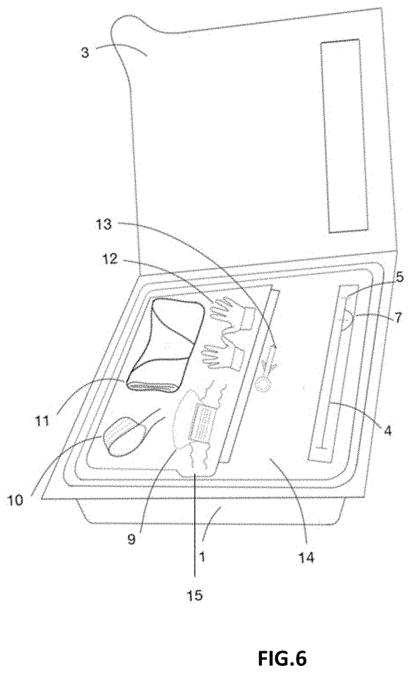

FIG. 6 illustrates the opening of the large cover (3) of the package or tray (1) giving access to the sterile supplies to perform the procedure and the drape (14). The sterile supplies include sterile mask (9), hat (10), gown (11), gloves (12) and sponge stick (13) with antiseptic solution such as alcoholic chlorohexidine and alcoholic iodine to perform skin antisepsis. The sterile supplies are located on top of a peel-off paper and plastic film cover (15) that is attached to the drape (14).

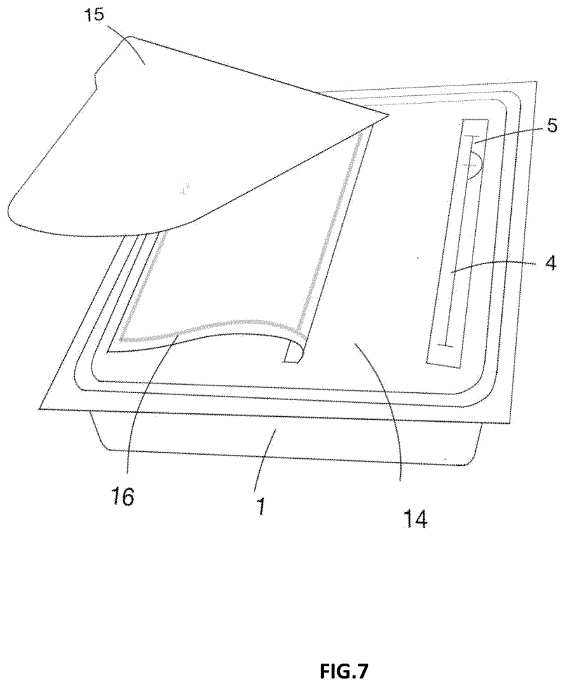

FIG. 7 illustrates the step after removal of the sterile supplies, the peel away cover (15) is removed and a plastic film (16) with adhesive substance is flipped over to provide sterile cover for the slots (4 & 5) where the probe, cable and tablet viewer were inserted.

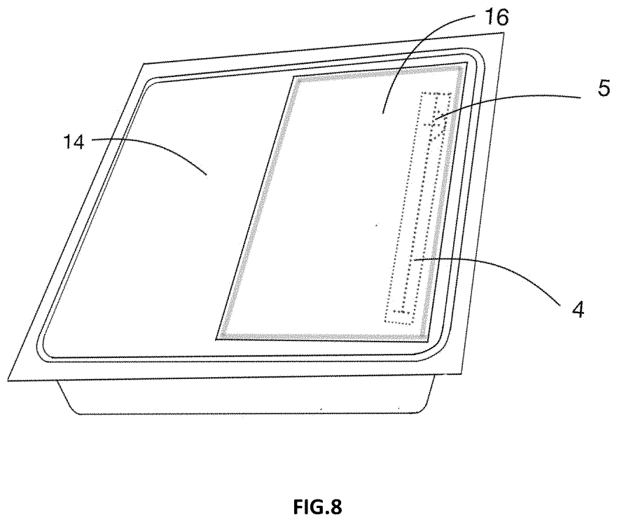

FIG. 8 illustrates the adhesive on the plastic film (16) facing the drape and provides complete sterile seal of the slots (4 & 5) and adjacent face where the probe and tablet had contact during the insertion. The seal also creates a physical closure for the opening of the respective slots (4 & 5) preventing the probe (6) and tablet (8) from dropping out of respective compartment during the manipulation of the drape (14).

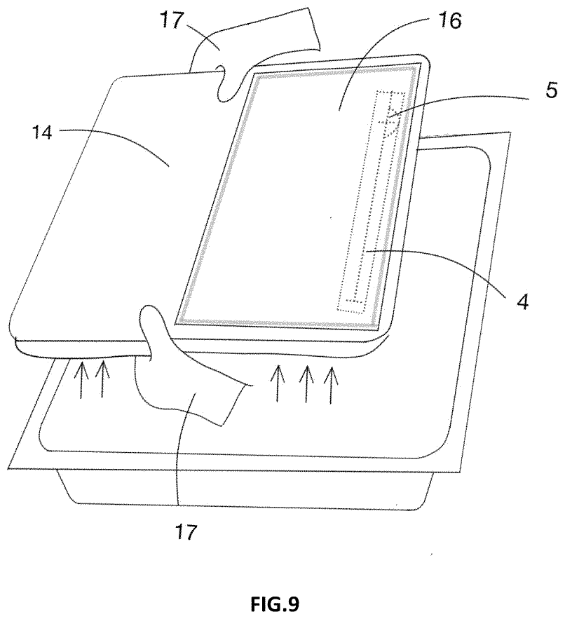

FIG. 9 illustrates the drape 14 with surface entirely sterile, being removed from the package or tray (1) by the sterile gloved hands (17) of the health care provider. The hands will hold each side and simultaneously lift the drape from package. The plastic film (16) covering the respective slots are facing up.

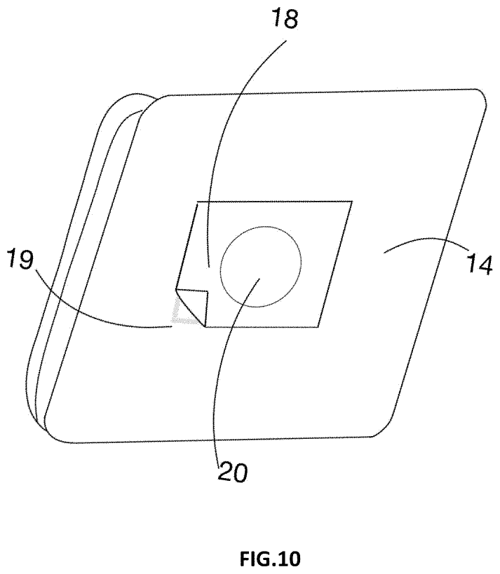

FIG. 10 illustrates the drape (14) surface that will be in contract with the patient. This surface is the opposite side of the drape displayed in all the previous figures. This surface of the drape contains a fenestration (20) with adhesive material of the edges covered by a peel off paper cover (18). The paper cover (18) should be removed before placement of the drape (14) on the patient's body surface.

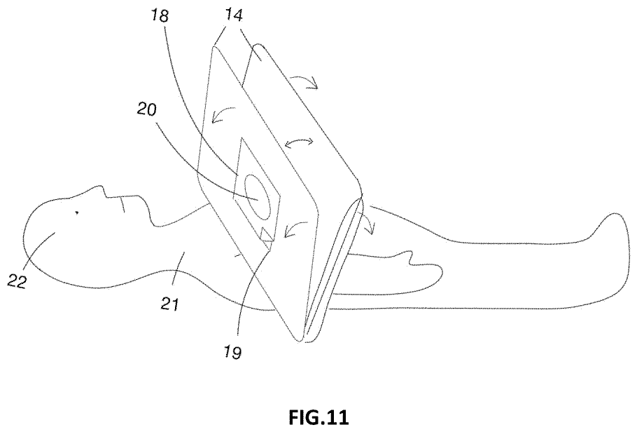

FIG. 11 illustrates the placement and opening of the drape (14) on the patient (22), the fenestration (20) should aim the final expected position of the procedure site (21). FIG. 11 is an example of central line catheterization of a neck vein, the internal jugular vein.

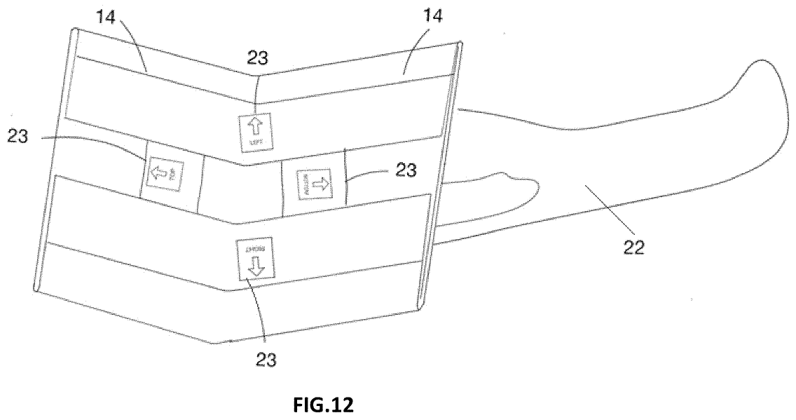

FIG. 12 illustrates the process of unfolding the drape (14), the edges of the drape contains respective labeling (23) from which direction the edge should be directed in relation to the patient's body. The labels on the edges contains arrows and words such as top, bottom, right and left. The labels also contain numbers to give directives for sequence and order in which the edges should be opened.

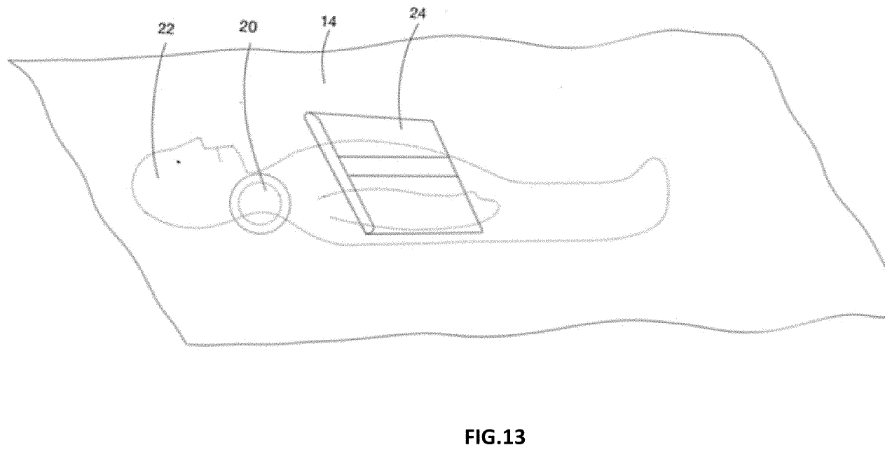

FIG. 13 illustrates the final position of the drape (14) on the patient (22), Note the fenestration (20) giving access to the patient's neck. The compartment (24) will give access to the probe, tablet and line placement supplies.

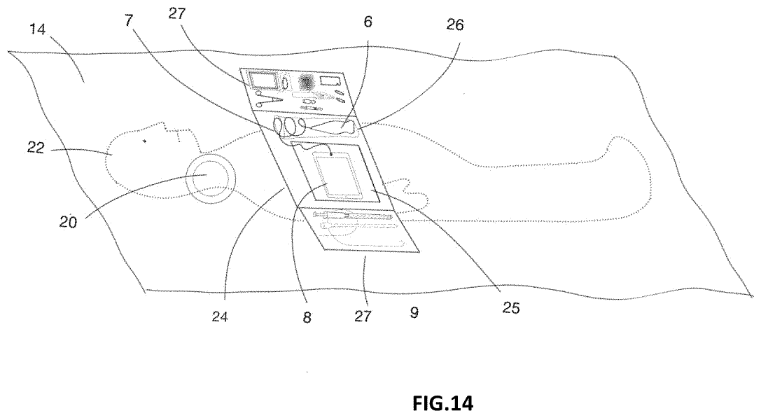

FIG. 14 illustrates the opening the compartment (24) on the drape that will give access to the probe (6), tablet (8), and central line supplies (27). The compartment for the tablet contains a clear and transparent film (25) that allows visualization of the images on the table display, the film also does not interfere with the tablet touch screen functionality. The probe compartment contains a flexible plastic cover (26) that allows placement of ultrasound gel on both sides of the cover. Rubber bands can be used to maintain close contact between the cover, gel and probe (6).

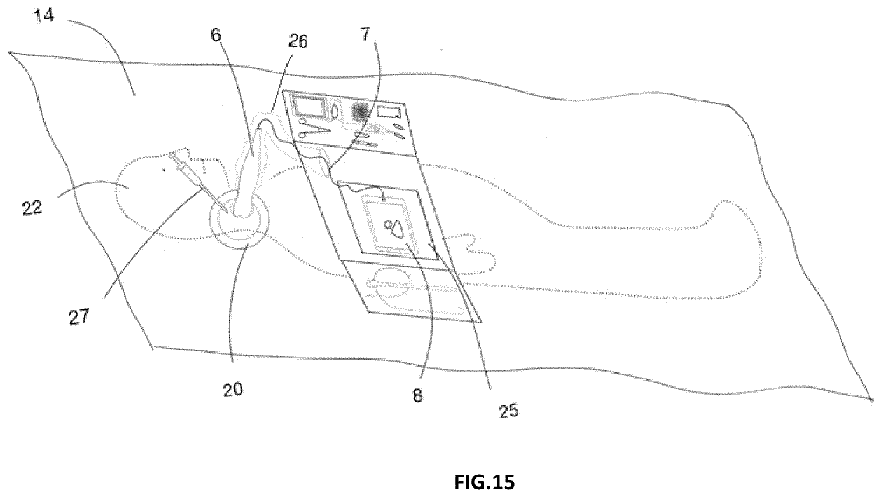

FIG. 15 illustrates the probe (6) placement on the patient's neck displaying the images of neck vessels on the tablet screen (8). Note the probe (6) and cable (7), are covered by flexible cover (26). Note the images of neck vessels displayed on the tablet (8) are visualized through the transparent film (25). The film (25) also prevents contamination of the sterile field. The ultrasound probe and tablet will be accessible inside the respective sterile compartments. When the ultrasound is appropriately positioned instrument package (24) containing the necessary supplies for the central line placement is opened to access intervention supplies. The instrument package (24) will be configured to be inside the drape (14) when the drape is folded for packaging of the kit. Once the kit is accessed and drape deployed the instrument package (24) will be positioned in a convenient location relative to the opening for ultrasound transducer. The ultrasound is used to guide placement of central line, the placement finalized and secured. The instrument package can contain one or more surgical or medical tools necessary to perform a particular medical or surgical procedure. This can be most any type of surgical or medical equipment or tools such as, for example, needle(s), cannula(s), sponge(s), wipe(s), sutures, forceps, scissor(s), and scalpels of various configurations. Other non-limiting examples of supplies or tools include sponges, syringes, anesthetics, needles, clamps, cannulas, vials, gauze pads, swabs, stapling devices, dissectors, and sutures to name a few.

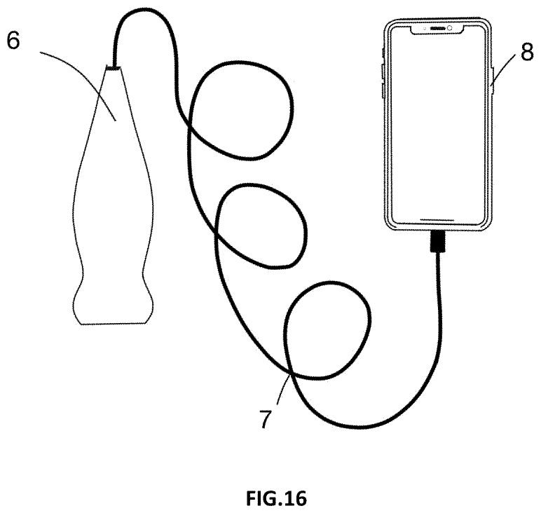

FIG. 16 illustrates the process of imaging using portable ultrasound probe (6), respective cable (7), and the viewing device (8), e.g., a smartphone used as a display screen or viewer for real time imaging. The use of such portable devices during sterile invasive procedures can be achieved with the embodiments herein described.

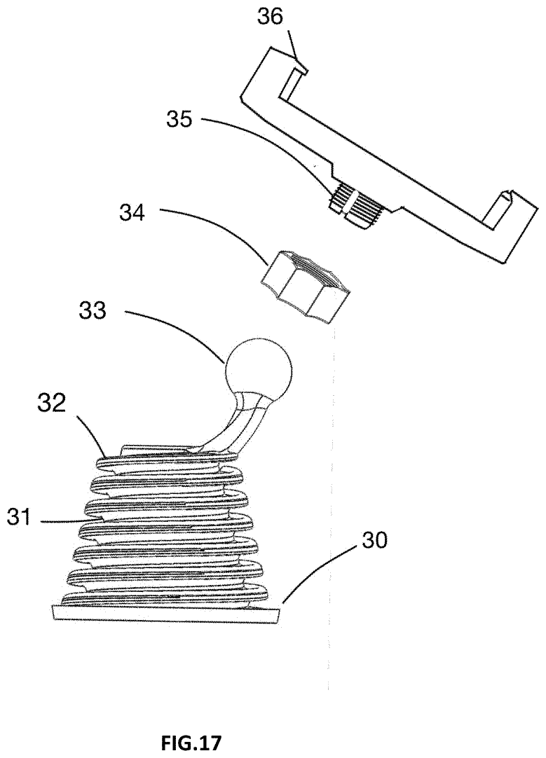

FIG. 17 illustrates the parts of one example of a mounting system that connects the probe (6), with the ultrasound system. A conical cup like part (30) in which the internal surface mirrors or conforms to the top or proximal external surface of the probe (6). The external surface of the part (30) contains a thread (32) for the placement and stabilization of a cable in a loop fashion. The dimension of the thread root (31) allows pressure fit and stabilization of the cable. A first attachment component (33), e.g., ball, attaches to a second attachment component (35), e.g, a socket, with, in this circumstance, a threaded bolt and nut (34) for tightening the mounting (36) for the smartphone.

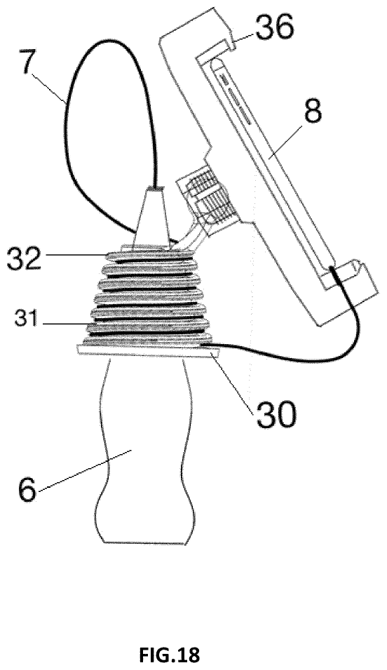

FIG. 18 illustrates the assembly of probe (6), smartphone (8), and mounting system (30 and 36). The thread (32) for the placement and stabilization of the cable in a loop fashion. The dimension of the thread root (31) allows pressure fit and stabilization of the cable (7).

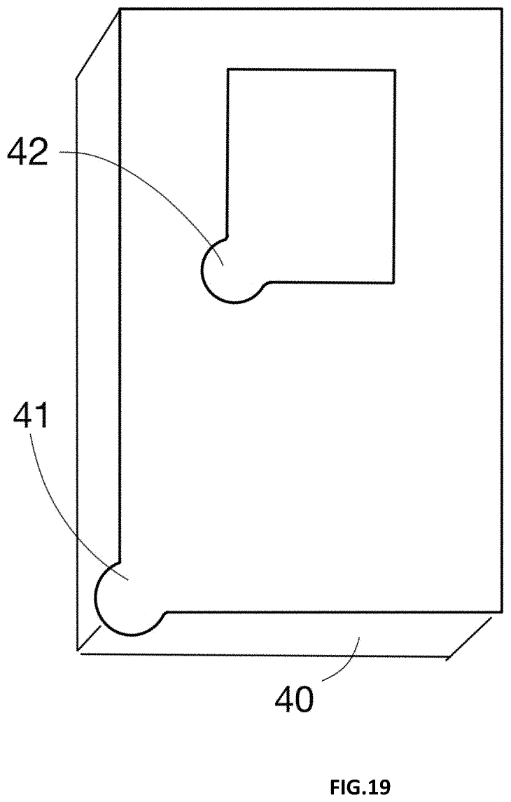

FIG. 19 illustrates the sterile package or tray (40) containing all the sterile supplies needed for the procedure (1), showing two covers, one small (42) covering a smaller area and one large (41) covering the remaining top package or tray.

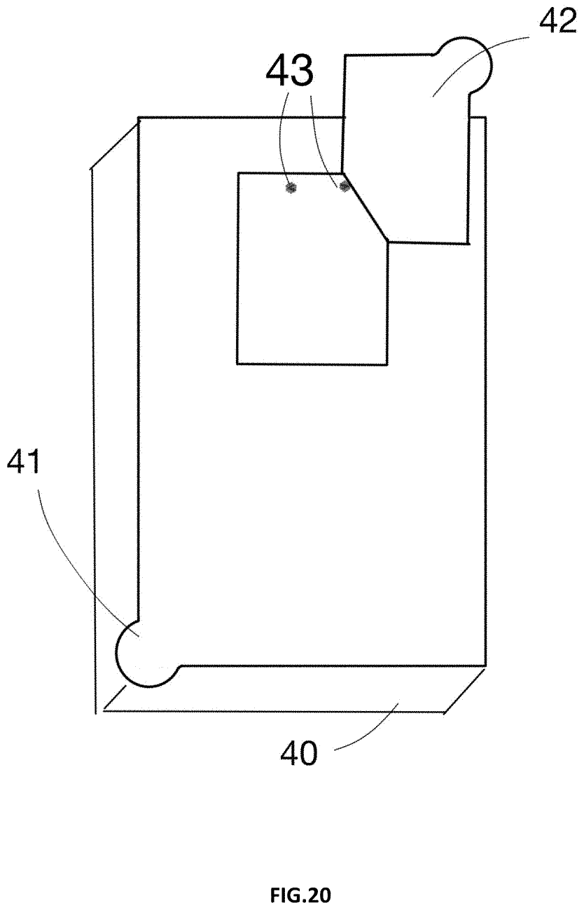

FIG. 20 illustrates the small cover (42) opening by peeling off from the large cover (41) of the tray (40). Two pins (43) for alignment and attachment to the back of the mounting (36) for the smartphone.

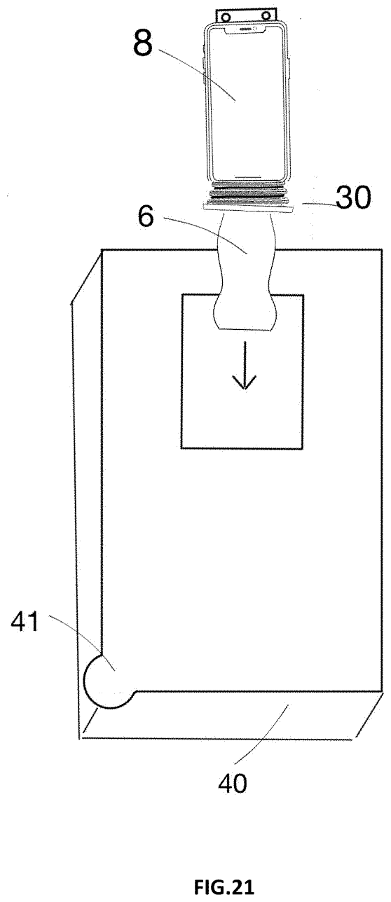

FIG. 21 illustrates the insertion of the assembly probe (6), smartphone and mount (30) inside the package through the package opening. The opening is created by removal of the small cover (42). Note the rest of package is sealed with the large cover (41)

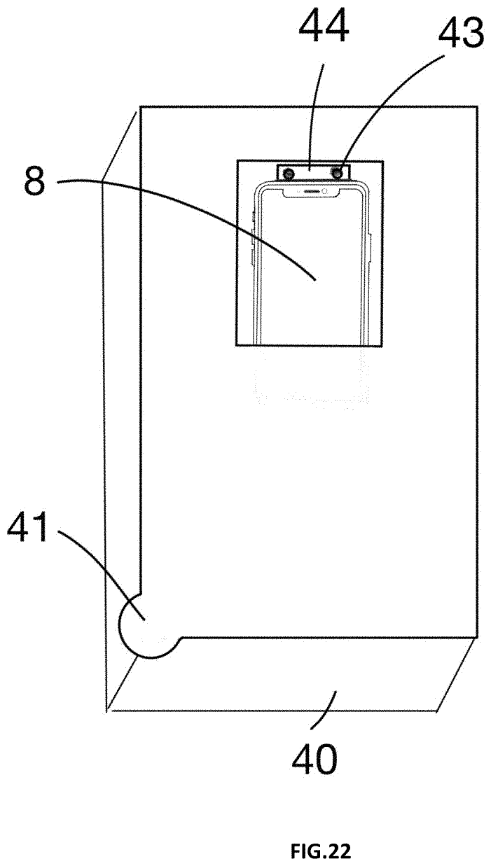

FIG. 22 illustrates the aligning plate on the back of the mounting system (44), is aligned and attached to the two alignment pins (43). Note the placement of the assembly inside the package (40), alignment and attachment of the plate (44) to the pins (43) is performed before opening of the large cover (41). Note the cover (41) is in place and not removed from the package (40).

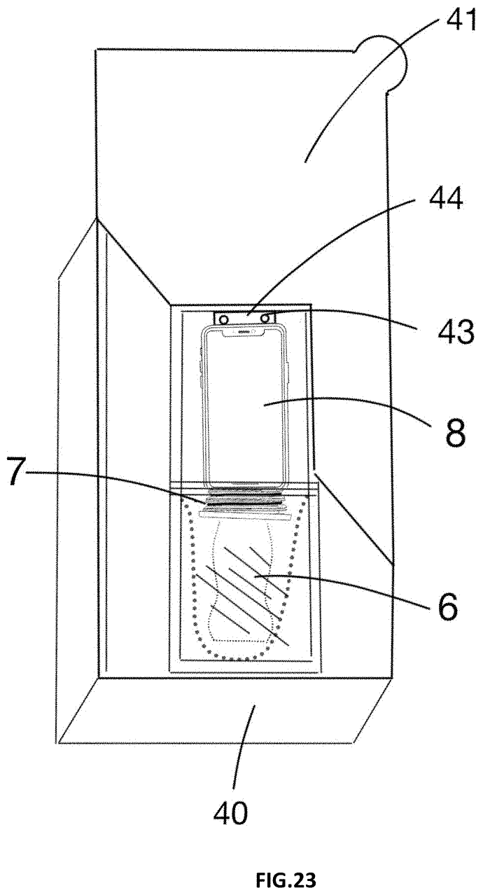

FIG. 23 illustrates the large cover (41) being peeled off from the package (40) showing the internal components of the package.

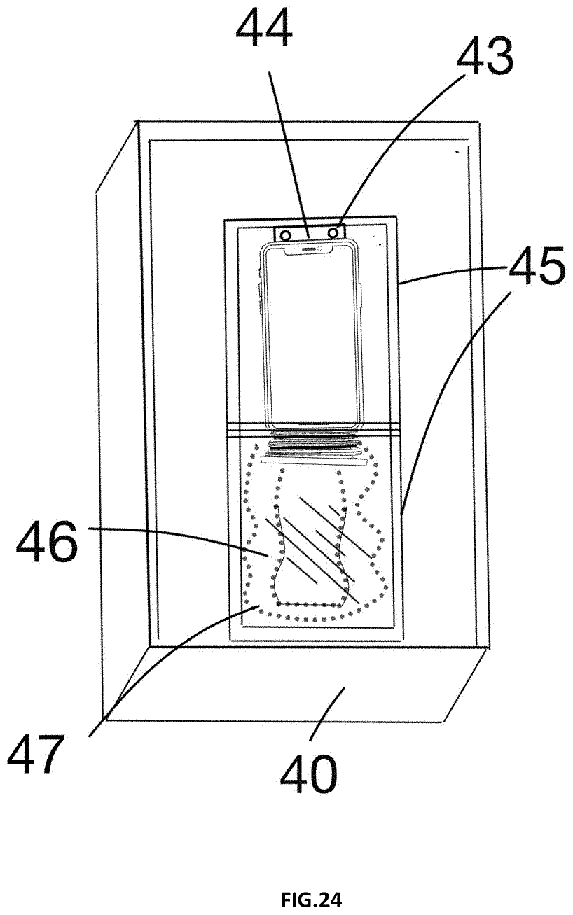

FIG. 24 illustrates the open tray (40) with the probe now inside the cover (47) and the top closure (45) in open position. The top cover (45) folds in a half, the first half is an open frame where the assembly will be inserted, and the second half is covered by a clear film (46).

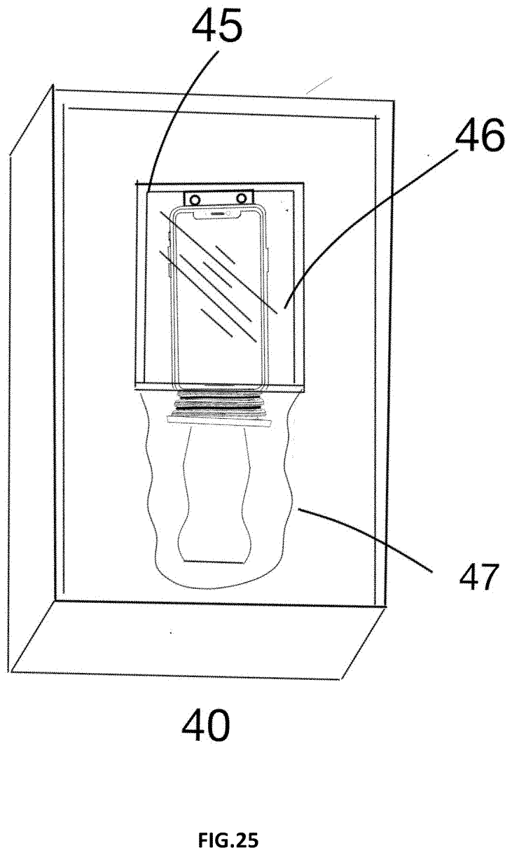

FIG. 25 illustrates the top cover (45) folded in a half closing the compartment. The external surface is all sterile sealing the non-sterile assembly on the inside.

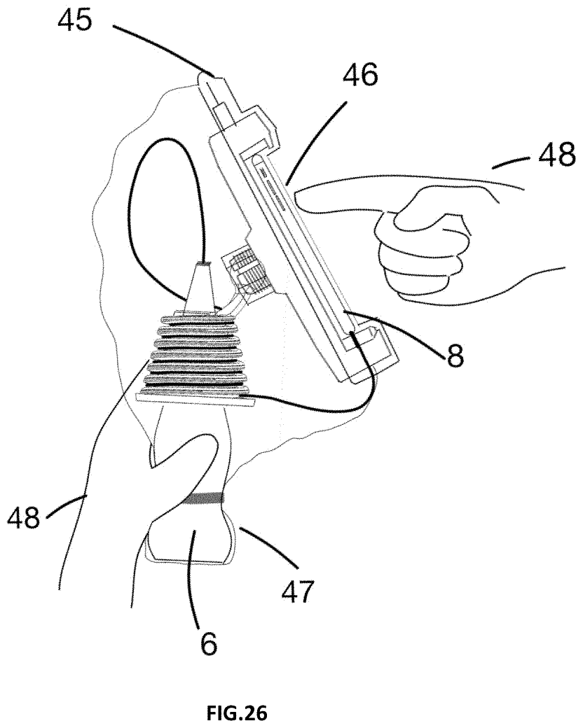

FIG. 26 illustrates the clear film (46) that allows visualization and touchscreen manipulation of the smartphone (8) by the provider using sterile gloves (48). Note the cover (45) for the probe (6) and the cover (45) for the smartphone (8) provides complete sealing of the assembly.

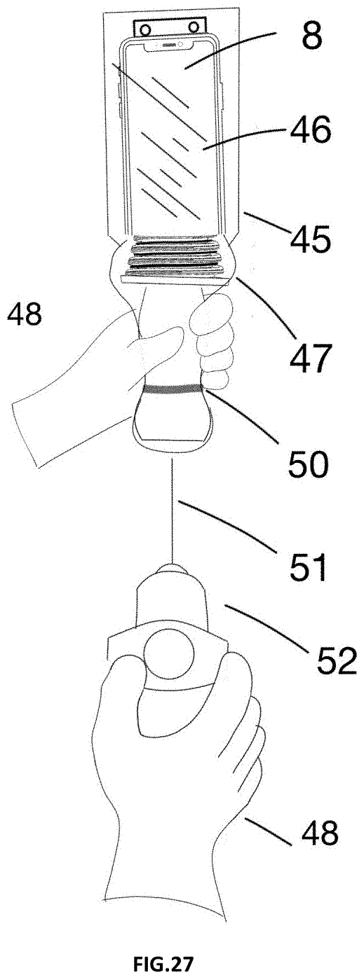

FIG. 27 illustrates an example of real time ultrasound guided procedure where the provider is using sterile gloves (48) holding the assembly with the left hand and the syringe (52) with needle attached (51) with the right hand. Note the cover (47) over the probe (6) and the cover (45) over the smartphone (8).

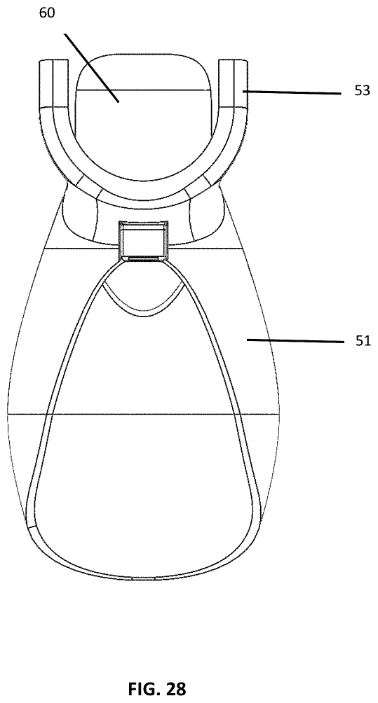

FIGS. 28 to 35 illustrate various views of an ultrasound adaptor for mounting a device on an ultrasound prove and a system incorporating the ultrasound adaptor (mounting system).

FIG. 28 illustrates the front view of mount or attachment system showing favorite embodiment in which the mount system fits over the ultrasound probe 51—Outer shell that fits over the probe 53—frame with slot for disk attachment. 60—Clip feature that gives the mount system additional feature to be used as a holster to facilitate transport of the portable probe.

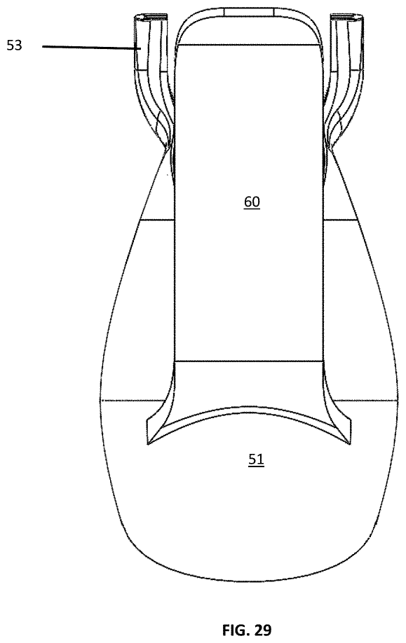

FIG. 29 illustrates the back view of mount or attachment 51—Outer shell that fits over the probe, 52-53—frame with slot for disk attachment. 60—Clip feature.

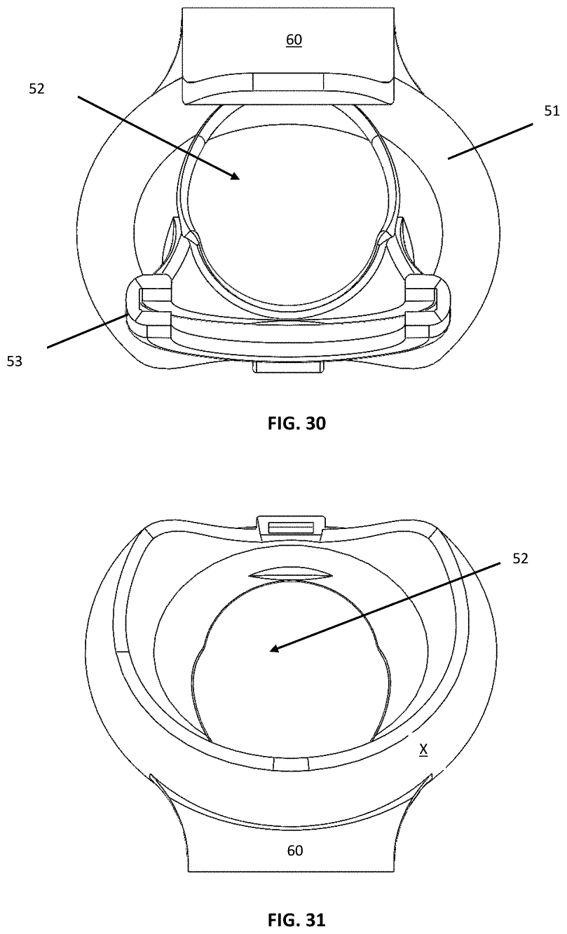

FIG. 30 illustrates the top view—of mount or attachment system showing favorite embodiment in which the mount system fits over the ultrasound probe 51—outer shell that fits over the probe, 52—central opening for probe placement, 53—frame with slot for disk attachment. 60—Clip feature.

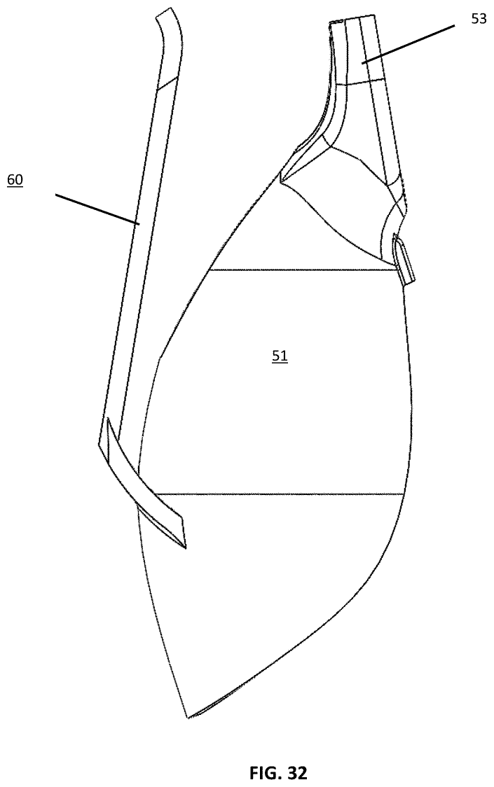

FIG. 31 illustrates the bottom view of mount or attachment system showing favorite embodiment in which the mount system fits over the ultrasound probe, 51—outer shell that fits over the probe, 52—central opening for probe placement, 53—frame with slot for disk attachment. 60—Clip feature.

FIG. 32 illustrates side view showing 51—Outer shell that fits over the probe, 53—frame with slot for disk attachment. 60—Clip feature.

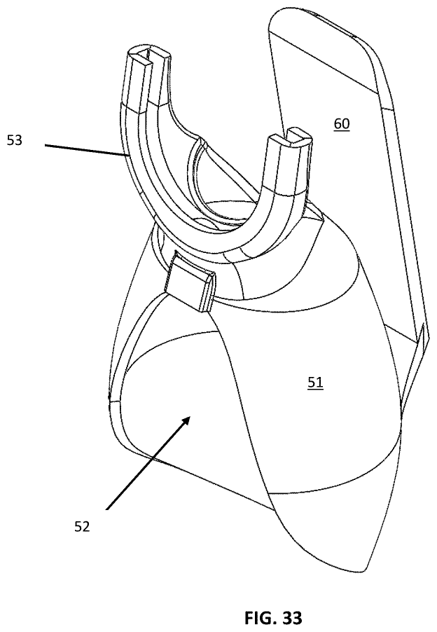

FIG. 33 illustrates the perspective view—51—outer shell that fits over the probe, 52—central opening for probe placement, 53—frame with slot for disk attachment. 60—Clip feature.

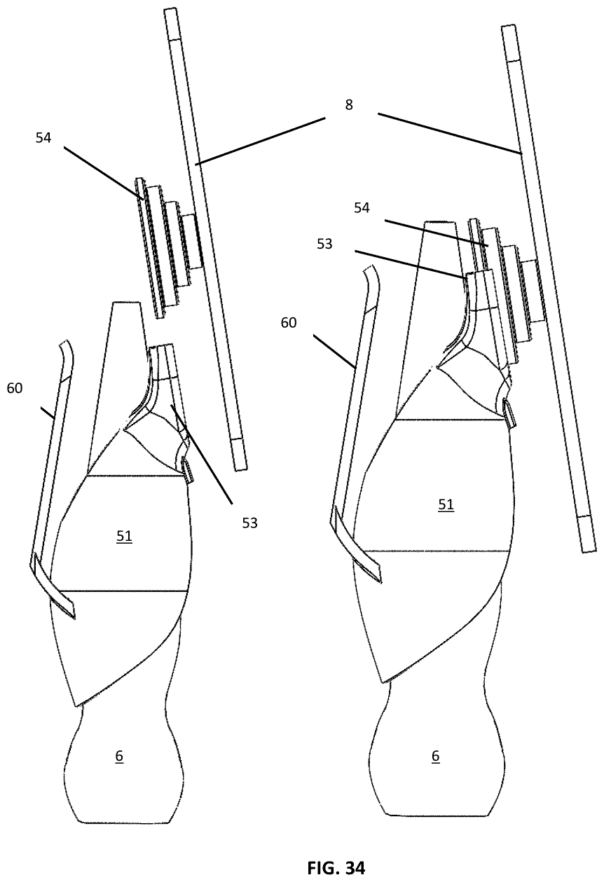

FIG. 34 illustrates the side view showing the assembly process using 6—the portable ultrasound probe, 8 the smartphone and 52 the mount system by inserting 54—the disk into the frame 53. On the left showing the alignment of the disk with the slot into the frame. On the right coupling the disk into the slot, to combine the smartphone with mounting system and portable ultrasound.

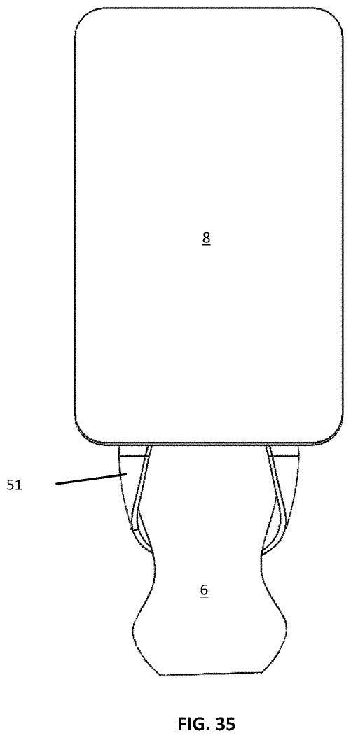

FIG. 35 illustrates the front view—showing the assembly 51—outer shell of attachment or mount system, 8—smartphone, ultrasound probe. Note the smartphone is now attached to the proximal end of the smartphone. The display of the smartphone provides continuous visualization of the ultrasound images and does not interfere with the scanning given by the transducer located on the distal end of the ultrasound probe.

The adaptor (e.g., FIG. 28) for a smartphone or other viewing device can be attached directly on the top or proximal end of the portable ultrasound probe (6) to provide a stable and continuous image, contain the cable, and not interfere with the scanning process. The viewing device's (8), e.g., a smartphone, position on top of the probe (6) puts the screen display on the same field of vision to perform real-time ultrasound-guided procedures. The attachment (53/54) encompasses physical or magnetic embodiment methods. The physical attachments include ball and socket, disk and slot, cradle, threading system, etc. The mounting system also can contain a magnetic connection between the smartphone (8) and the probe (6). The mounting system allows smartphone tilt, rotation, and different portrait orientations. The mount feature contains a tread, groove, or spool feature to hold the cable and prevent contamination.

A sterile cover is shaped to fit or conform to the assembly (probe (6), mount (53/54), and smartphone (8)), inside a clear sterile cover that allows screen visualization and touch screen manipulation of the smartphone display. The final assembly (portable probe (6), smartphone (8), and adaptor, with or without a probe cover) is entirely handheld for scanning using one hand.

1. A kit comprising a procedural tray configured for assembling and sterile use of a smartphone, a tablet, a portable imaging system, or wireless technologies during needle guided interventions or surgery.

2. A sterile drape comprising compartments configured to allow visualization and sterile manipulation of smartphone, tablet, or portable imaging systems during a procedure, the sterile drape configured and aligned with a package tray for opening and sealing of the drape in sterile fashion.

3. (canceled)

4. A mount system for a smartphone to be attached directly on the top of a portable ultrasound probe, the mount system having an adaptor configured to fit over the proximal end of an ultrasound probe, the adaptor having a reversable attachment portion that is configured to attach to a viewing device and then be reversibly coupled to the adaptor.

5. The system of claim 4, wherein the adaptor is coupled to the attachment portion by a disk/slot, ball/cup, cradle, threaded, or magnetic coupling.

6. The system of claim 4, wherein the system is configured to allow tilting, rotation, and orientation of the viewing device.

7. The system of claim 4, wherein the adaptor has a belt or waistband clip allowing the mount system to work interchangeably as a holster for transport of the portable probe.

8. The system of claim 4, wherein the adaptor comprises a tread, groove, or spool configured to hold cabling.

9. The system of claim 4, comprising a clear sterile cover that is positioned over and contains a probe, mount, and smartphone assembly and is configured to allow screen visualization and touch screen manipulation of the viewing device when in use.

10. (canceled)

11. (canceled)
